(19) United States
(12) Patent Application Publication
(10) Pub. No.: US 2016/0108433 A1
(43) Pub. Date: Apr. 21, 2016

(54) SYSTEMS, APPARATUS, AND METHODS FOR DROPLET-BASED MICROFLUIDICS CELL PORATION

(71) Applicant: DUKE UNIVERSITY, Durham, NC (US)

(72) Inventors: Richard Fair, Durham, NC (US); Andrew Madison, Durham, NC (US); Matthew Royal, Durham, NC (US)

(21) Appl. No.: 14/979,705

(22) Filed: Dec. 28, 2015

Related U.S. Application Data
(63) Continuation of application No. PCT/US2014/044526, filed on Jun. 27, 2014.
(60) Provisional application No. 61/840,057, filed on Jun. 27, 2013.

(51) Int. Cl. C12N 15/87; B01L 3/00

(52) U.S. Cl. CPC C12N 15/87; B01L 3/502715; B01L 2400/0427; B01L 2300/165

(57) ABSTRACT

The present disclosure provides systems, apparatus, and methods that include an electrowetting-on-dielectric microfluidic apparatus for effecting poration of cells in a droplet.

### CROSS REFERENCE TO RELATED APPLICATIONS

This application is a Continuation Application of International Patent Application No. PCT/US2014/044526 filed on Jun. 27, 2014, which claims priority to U.S. provisional patent application number 61/840,057 filed Jun. 27, 2013, the disclosures of which are incorporated herein by reference in their entireties.

### FEDERAL FUNDING LEGEND

The invention was made with government support under Grant No. HR0011-12-C-0057 awarded by the Defense Advanced Research Projects Agency. The Government has certain rights in the invention.

### TECHNICAL FIELD

The presently disclosed subject matter relates to systems and methods for cell poration using droplet-based microfluidics.

### BACKGROUND

Cell transfection can be achieved by the transfer of synthetic DNA fragments through temporary cell membrane pores that are introduced by the application of an external electric field. The applied electric field sets up a trans-membrane potential sufficient to breakdown the cell wall and electrophoretically pushes negatively charged molecules, such as DNA, into the cell. This method is known as electroporation and has been well documented since the early 1980s.

The bilayer structure of a cell membrane is a dielectric. Accordingly, a trans-membrane potential is induced when the membrane is exposed to an electric field. For most cells, if the trans-membrane potential exceeds 1V, the membrane becomes porous, and thus permeable to extracellular materials. Hence, electroporation involves the transient increase in membrane permeability through application of an appropriate electrical pulse. With increasing electric field strengths, the cell membrane breaks down, becomes irreversible, and the likelihood of cell lysis, which leads to cell death, increases.

Under typical electroporation conditions, all the cells between the electrodes which are generating the electric field are subjected to some electric field level, resulting in cell membrane electroporation, cell lysing, or no effect. This method is known as bulk electroporation. Bulk electroporation represents the average response of all cells to the applied electric field between the electrodes, while the reaction of individual cells to the applied electric field may vary substantially. Thus, the cells are placed in an electric field that varies spatially, and only those cells that are in the region of a certain field strength porate, while the rest do not. To increase the number of porated cells, the electric field is increased, resulting in more transfected cells but also more cells that porate to the point of lysis, and are thereby killed. In addition, conditions for transfection need to be found for each cell type, because optimal transfection conditions depend on cell size, buffer chemistry, and DNA fragment size. Thus, low cell viability and low transfection rates are trademarks of conventional bulk electroporation.

An alternative method of transfecting cells is the use of “sonoporation,” which uses some form of ultrasonic cavitation to generate transient pores in cell membranes, allowing drug, DNA, and antibody delivery into the cell [Wu J, and Nyborg W L (2008) Adv, Drug Delivery Reviews, vol. 60, pp.1103-1116]. Efficient sonoporation has been obtained with transmitted ultrasound frequencies in the 0.5-4 MHz range. The mechanism of molecular translocation into cells via sonoporation is not well understood, but it is likely that microbubbles under ultrasound insonation are responsible [Pichon C, et al. (2008) J. Experimental Nanoscience, vol. 3, pp. 17-40]. Cells in contact with a microbubble undergo deformation [Van Wamel A, et al. (2004) Ultrasound Med. Biol. Vol. 30, pp.1255-1258]. At low acoustic pressure, the microbubble-cell interaction generates a mechanical compressive stress. Similar to conventional bulk electroporation methods, sonoporation methods are similarly limited by low cell viability and low transfection rates.

Thus, there remains an unmet need for systems and methods for improved cell tranfection efficiency. The present disclosure provides such systems and methods.

### SUMMARY

In one embodiment of the present disclosure, an electrowetting-on-dielectric microfluidic apparatus for cell poration is provided including: a first plate; a second plate spaced-apart from the first plate and defining a flow channel therebetween for receiving a droplet having cells therein; an acoustic energy generator in communication with the first plate for providing sonoporation to cells within a droplet received in the flow channel; a resilient member in communication with the acoustic energy generator and in communication with a droplet received in the flow channel to provide acoustic streaming and sonoporation to cells within a droplet; and an electric field generator in electrical field communication with the flow channel for providing electroporation to cells within a droplet received in the flow channel.

In one embodiment of the present disclosure, a system is provided including: an electrowetting-on-dielectric microfluidic apparatus for cell poration that includes: a first plate; a second plate spaced-apart from the first plate and defining a flow channel therebetween for receiving a droplet having cells therein; an acoustic energy generator in communication with the first plate; a resilient member in communication with the acoustic energy generator and in communication with a droplet received in the flow channel; and an electric field generator in electrical field communication with the flow channel; and a control module configured for: controlling the acoustic energy generator to apply energy to the apparatus for providing acoustic streaming and sonoporation to cells within a droplet; and controlling the electric field generator to apply an electric field for providing electroporation to cells within a droplet.

In one embodiment of the present disclosure, a method is provided including: applying an acoustic energy field to a resilient member in communication with a droplet having cells therein to induce an ultrasonic vibration in the resilient member to effect sonoporation of the cells.

In one embodiment of the present disclosure, a method is provided including: in a spaced-apart plate arrangement defining a flow channel therebetween, applying an acoustic energy field to a resilient member in communication with a droplet having cells therein to induce an ultrasonic vibration in the resilient member for providing acoustic streaming and sonoporation of the cells; and applying an electric field to the droplet having cells therein to effect electroporation of the cells.

In one embodiment of the present disclosure, an electrowetting-on-dielectric microfluidic apparatus for cell poration is provided including: a first plate; a second plate spaced-apart from the first plate and defining a flow channel therebetween for receiving a droplet having cells therein; an acoustic energy generator in communication with the first plate for providing sonoporation to cells within a droplet received in the flow channel; and a resilient member in communication with the acoustic energy generator and in communication with a droplet received in the flow channel to provide acoustic streaming and sonoporation to cells within a droplet.

In one embodiment of the present disclosure, a system is provided including: an electrowetting-on-dielectric microfluidic apparatus for cell poration that includes: a first plate; a second plate spaced-apart from the first plate and defining a flow channel therebetween for receiving a droplet having cells therein; an acoustic energy generator in communication with the first plate; a resilient member in communication with the acoustic energy generator and in communication with a droplet received in the flow channel; and a control module configured for: controlling the acoustic energy generator to apply energy to the apparatus for providing acoustic streaming and sonoporation to cells within a droplet.

In one embodiment of the present disclosure, an electrowetting-on-dielectric microfluidic apparatus for cell poration is provided including: a first plate; a second plate spaced-apart from the first plate and defining a flow channel therebetween for receiving a droplet having cells therein; an immersion transducer extending from the first plate into the flow channel to provide acoustic streaming and sonoporation to cells within a stationary droplet received in the flow channel; and an electric field generator in electrical field communication with the flow channel for providing electroporation to cells within a stationary droplet received in the flow channel.

In one embodiment of the present disclosure, a system is provided including: an electrowetting-on-dielectric microfluidic apparatus for cell poration that includes: a first plate; a second plate spaced-apart from the first plate and defining a flow channel therebetween for receiving a droplet having cells therein; an immersion transducer extending from the first plate into the flow channel; and an electric field generator in electrical field communication with the flow channel; and a control module configured for: controlling the immersion transducer to apply energy to the apparatus for providing acoustic streaming and sonoporation to cells within a stationary droplet; and controlling the electric field generator to apply an electric field for providing electroporation to cells within a stationary droplet received in the flow channel.

### BRIEF DESCRIPTION OF THE DRAWINGS

The foregoing aspects and other features of the invention are explained in the following description, taken in connection with the accompanying drawings.

FIG. 1 is a schematic diagram of a system according to one or more embodiments of the present disclosure in which a droplet-based microfluidics apparatus is used to effect cell poration.

FIG. 2 is a front view of an electrowetting-on-dielectric microfluidic apparatus for cell poration according to one or more embodiments of the present disclosure.

FIG. 3 is a front view of an apparatus for cell poration according to one or more embodiments of the present disclosure.

FIG. 4 is a front view of an apparatus for cell poration according to one or more embodiments of the present disclosure.

FIG. 5 is a front view of an apparatus for cell poration according to one or more embodiments of the present disclosure.

FIG. 6 is a top view of the apparatus shown in FIG. 5.

FIG. 7 is a schematic flow diagram of a method according to one or more embodiments of the present disclosure.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

The term “transfection” is used herein as the deliberate introduction of nucleic acids into any cell type, not only to eukaryotic cells as in the conventional definition.

Unless otherwise defined, all technical terms used herein have the same meaning as commonly understood by one of ordinary skill in the art to which this disclosure belongs.

The presently disclosed subject matter addresses the major drawbacks of bulk electroporation in a droplet, which are 1) transfection of a limited number of cells in a droplet due to the application of a large electric field using electrodes immersed within the droplet's liquid volume resulting in each cell in the electrode space being subjected to an unknown field due to shielding by other cells in that space, and 2) transfection of a limited number of cells resulting from the limited exposure volume within the droplet to the required field for electroporation due to the cells being stationary and either grounded or floating metal field plates in the droplet-based microfluidic device.

Software automated genomic engineering (SAGE) enables genetic modification of cells on a fluidic platform that implements a multiplex automated genomic engineering (MAGE) process. Electrowetting-on-dielectric (EWD) digital microfluidics is well-suited for SAGE because of its inherent reconfigurability, small reagent volumes, and parallel processing capability. Cell transfection has been achieved by the transfer of synthetic DNA fragments through temporary cell membrane pores that were introduced by the application of an external electric field. The applied electric field sets up a trans-membrane potential sufficient to breakdown the cell wall and electrophoretically pushes negatively charged molecules, such as DNA, into the cell. The bilayer structure of a cell membrane is a dielectric. Accordingly, a trans-membrane potential is induced when the membrane is exposed to an electric field. For most cells, if the trans-membrane potential exceeds 1 Volt, the membrane becomes porous, and thus permeable to extracellular materials. Hence, electroporation involves the transient increase in membrane permeability through application of an appropriate electrical pulse. With increasing electric field strengths, cell membrane breaks down, becomes irreversible, and the likelihood of cell lysis, which leads to cell death, increases.

In the bulk electroporation to enable genome-wide engineering of cell populations by introducing synthetic DNA molecules into living cells, the cells were contained in 350 nL droplets on the EWD platform. Existing prior art droplet-based microfluidics systems do not allow for cell transfection. Thus, a system, apparatus, and method for bulk transfection of cells under software control was developed by the inventors. Insertion of engineered nucleic acid bases into the cellular genome of Eschericia coli cells was used to exemplify the system. The exemplary bench top MAGE system has a footprint of about 35 square feet and utilizes robotic pipetting, heating/cooling plates, and an array of cuvettes that are each comprised of two electrodes that “shock” a cell suspension with a high electric field. The fluid in the cuvette is stationary (no mixing) during bulk electroporation.

There are more than a million cells in each 350 nl droplet. Thus, popular single-cell transfection methods cannot be used. However, the droplet-based electrowetting device was modified beyond the standard electrode-pair structure to perform non-optimal bulk transfection of many cells. Two-plate structures for electroporating cells in solution in a stationary droplet were integrated into the MAGE system. Custom machined electroporation electrodes spaced 1 mm apart were inserted into the top plate of the DMP and pulsed using a commercial electroporation pulse delivery system. In this system, a droplet was held stationary in an EWD actuator while the electrodes inserted through the top plate of the actuator were energized.

Typical electric field strengths required for electroporation in these studies are 1800 V/mm, delivered in a pulse with a decay rate of 6 msec. Under these conditions, all the cells between the electrodes are subjected to some electric field level, resulting in cell membrane electroporation, cell lysing, or no effect. This method is known as bulk electroporation. Bulk electroporation in a droplet represents the average response of all cells to the applied electric field between the electrodes, while the reaction of individual cells to the applied electric field may vary substantially. Thus, the cells are placed in an electric field that varies spatially, and only those cells that are in the region of a certain field strength porate, while the rest do not. To increase the number of porated cells, the electric field is increased, resulting in more transfected cells but also more cells that porate to the point of lysis, and are thereby killed. Bulk electroporation is typically capable of processing thousands to tens of millions of cells simultaneously. High voltages are usually applied to bulk samples, and this is usually associated with the excessive Joule heating, bubble generation, electrode contamination, nonhomogeneous electric field across the different cells, low viability and low electroporation efficiency. Above all, the obtained results of bulk electroporation represent the average response of all cells to the applied electric field, while the individual reaction of cells to the applied electric field may differ substantially. In addition, ideally, conditions for transfection need to be found for each cell type, because optimal transfection conditions depend on cell size, buffer chemistry, and DNA fragment size. Thus, low cell viability and low transfection rates are limitations of bulk electroporation.

A transfection efficiency of up to 2% was achieved in this process (evaluated as the ratio of transfected cells to survived cells) while maintaining fluid transport capability in the EWD system. Specifically, of 7 million EcNR2 E. Coli cells introduced in a droplet containing a pZE21-GFP plasmid, on average only 625,000 cells survived electroporation. In addition, on average only 14,000 cells were transfected. While improvements are foreseeable in the level of transfection efficiency with modifications to the electrode design and/or experimental conditions, the percentage of a droplet's volume that actually is exposed to the required 1800 V/mm field is limited to a relatively small amount (<20%) due to the presence of metal actuation surfaces in the bottom and top plates of the EWD devices. The result is that a relatively small number of the cells in the droplet volume are electroporated, transfected, and remain viable, a limitation of the bulk electroporation

An alternative method of transfecting cells is the use of “sonoporation”, which uses some form of ultrasonic cavitation to generate transient pores in cell membranes, allowing drug, DNA, and antibody delivery into the cell. Efficient sonoporation has been obtained with transmitted ultrasound frequencies in the 0.5 to 4.0 MHz range. The mechanism of molecular translocation into cells via sonoporation is not well understood, but it is likely that microbubbles under ultrasound insonation are responsible. Cells in contact with a resilient member, such as a microbubble, undergo deformation. At low acoustic pressure, the microbubble-cell interaction generates a mechanical compressive stress. Sonoporation constitutes a transfer method that is low in toxicity, easy to implement, adaptable to different cell types, and suitable for in vivo situations.

In one aspect, the presently disclosed subject matter provides systems, methods, and apparatus for integrating sonoporation and electroporation into a droplet-based microfluidic device (electrosonic poration). In one aspect, the presently disclosed subject matter provides systems, methods, and apparatuses for integrating sonoporation alone into a droplet-based microfluidic device. In one embodiment of the present disclosure, a resonating microbubble in a top plate of a EWD device, which is driven by ultrasonic energy, can induce an acoustically focused flow within the droplet, thereby directing cells to the surface of a resilient member, where sonoporation of cells occurs. The induced flow is circular in that it can direct cells from all parts of the droplet volume to the locally resonating member, thus allowing full-droplet volume transfection of cells. The acoustic streaming flow focuses cells into a stream that is on the order of the diameter of the resonating member. Electroporation electrodes placed below the top plate and spaced apart approximately by the diameter of the resilient member can bound the streaming flow of cells. An electric field may be applied between the electrodes of sufficient magnitude to cause electroporation of cells being carried in the narrow, streaming flow. As a result, sonoporation and electroporation, either individually or in combination can be applied to transfect the majority of cells in a droplet in a controlled and efficient manner.

Droplet-based microfluidics is a leading contender for a cell manipulation platform capable of qualitatively changing the scope of biological manipulations. Such manipulations include engineering industrial or pharmaceutical cultures to be resistant to contamination, which is a major problem for large-scale bioprocessing facilities, or adding non-natural amino acids to allow the bioproduction of chemicals and materials at a reduced cost. At the heart of cell manipulation processes is the efficient delivery of exogenous cargos (such as nucleic acids, proteins, and small drugs) into cells, which is being aggressively pursued to increase our understanding of gene regulation mechanisms and to yield promising pharmaceutical and/or medical benefits in drug discovery, cancer treatment, and regenerative medicine.

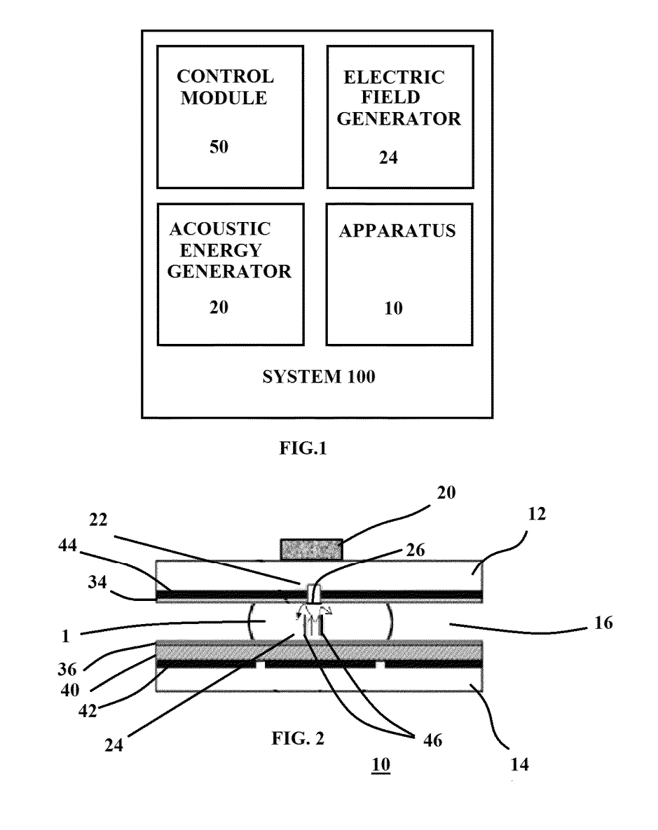

FIG. 1 is a diagram of a system (100) according to one or more embodiments of the present disclosure in which droplet-based microfluidics is used to effect cell poration. Such cell poration on a droplet-based microfluidics apparatus is a cell manipulation platform cabable of qualitatively changing the scope of biological manipulations. The system shown in FIG. 1 includes an electrowetting-on-dielectric microfluidic apparatus (10) for poration of cells within a droplet, an acoustic energy generator (20), an electric field generator (24) and a control module (50) configured for controlling the acoustic energy generator (20) to apply energy to the apparatus (10) for providing acoustic streaming and sonoporation to cells within a droplet, and controlling the electric field generator (24) to apply an electric field for providing electroporation to cells within a droplet.

An electrowetting-on-dielectric microfluidic apparatus for cell poration (10) according to one or more embodiments of the present disclosure is shown in FIG. 2. The apparatus (10) can include a first plate (12) and a second plate (14) spaced-apart from the first plate (12) and that defines a flow channel (16) therebetween for receiving a droplet (1) containing cells to be transfected. The apparatus (10) can include the acoustic energy generator (20) in communication with the first plate (12) for providing sonoporation to cells within a droplet (1) received in the flow channel (16). The apparatus (10) can include a resilient member (22), which is in communication with the acoustic energy generator (20) and in communication with a droplet (1) received in the flow channel (16) to provide acoustic streaming and sonoporation to cells within a droplet (1). The resilient member (22) can be a deformable membrane (26). The apparatus (10) can include the electric field generator (24) in electrical field communication with the flow channel (16) for providing electroporation to cells within a droplet (1) received in the flow channel (16). A droplet (1) received in the flow channel (16) of the apparatus (10) can be a stationary droplet. In this manner, the droplet (1) is stationary during the poration process. The droplet (1) may be held in a stationary position before and after the application of an electroporation pulse by the application of a DC or AC voltage on a patterned metal electrode (42) beneath the droplet. However, during the electroporation pulse, the electrode (42) beneath the droplet (1) must be temporarily electrically disconnected to avoid damaging the control electrode's electronic circuits.

In the apparatus (10) shown in FIG. 2, acoustic streaming is used to create a focused flow stream of cells within a droplet (1) that passes between spaced-apart electrodes (46) provided within the flow channel (16). The flow of cells in the droplet (1) is such that cells from substantially the full volume of the droplet (1) are made to pass through the spaced-apart electrodes (46), providing a localized, uniform electric field to be applied to cells as they pass through the space between spaced-apart electrodes (46). In addition, the flow of cells is directed to the resilient member (22), which results in ultrasonic cavitation and sonoporation of cells at the interface of the droplet (1) and the resilient member (22).

The resilient member (22) of the apparatus (10) can be a deformable membrane (26) carried by the first plate (12) and in communication with a droplet (1) received in the flow channel (16). In this manner, the membrane (26) is responsive to acoustic, ultrasonic, or other energy provided by generator (20) and vibrates in response to external energy fields. The vibration of the membrane (26) may mimic a flap valve such that the membrane (2) vibrates generally upwardly and downwardly, with such vibration imparting vibration to the droplet (1) when in communication with membrane (26).

In one or more embodiments, the electrodes (46) can be spaced-apart a distance generally corresponding to a width of the resilient member (22).

The first plate (12) of the apparatus (10) can include a hydrophobic layer (34) on a metal film. The hydrophobic layer (34) can include a Teflon® layer.

The second plate (14) of the apparatus (10) can include a hydrophobic layer (36) on a dielectric film (40) on a patterned metal electrode (42). The hydrophobic layer (36) can include a Teflon® layer. The dielectric film (40) can include a Parylene® layer.

The first plate (12) of the apparatus (10) can include a ground electrode (44). When the first plate (12) includes a ground electrode (44), the second plate (14) can include a control electrode (42).

The acoustic energy generator (20) of the apparatus (10) can be a piezoelectric disk translator, or any other device capable of providing vibratory movement of the resilient member (22).

The apparatus (10) can further include a function generator in communication with the acoustic energy generator (20). In this manner, the acoustic energy generator (20) may have any desired input function if it is determined that certain function signals may be advantageously provided. Similarly, the apparatus (10) can include an acoustic amplifier in communication with the acoustic energy generator (20).

FIG. 3 is a front view of an electrowetting-on-dielectric microfluidic apparatus (10) for cell poration according to one or more embodiments of the present disclosure. The apparatus (10) depicted in FIG. 3 shares many features as the apparatus illustrated in FIG. 2, and where the same features are shown, the description of those features as it relates to FIG. 2 may also apply to the features of the apparatus (10) shown in FIG. 3. As illustrated, apparatus (10) can be a bubble (30) partially enclosed by a recess (32) defined by the first plate (12). The bubble (30) is in communication with a droplet (1) received in the flow channel (16) when the droplet (1) is received proximal the bubble (30) during a poration process. In the apparatus (10) depicted in FIG. 3, localized acoustic streaming of liquid inside the droplet (1) is accomplished by a trapped or partially encapsulated bubble (30) in the top plate (12) of the apparatus (10) that is set into vibration by the sound field generated by the externally mounted ultrasound transducer (20) (e.g. a PZT disk). In the illustrated embodiment, the trapped bubble (30) sits in a cavity that can be drilled into the top plate (12) to the desired dimensions, shape, or other configuration.

The diameter of the trapped bubble (30) generally controls the width of the flow stream of cell movement in droplet (1), as shown in FIG. 3. The bubble (30), which may be an encapsulated air bubble or may be of other gaseous or fluid makeup, in a liquid medium can act as an actuator when the bubble undergoes resonant vibration in a sound field, since the surface of the bubble (30) behaves like a vibrating membrane. Frictional forces generated at the air/liquid interface induce a bulk fluid flow around the bubble (30), which may be referenced herein as cavitation microstreaming or acoustic microstreaming. The frequency of resonance is determined by the bubble radius, the hydrostatic pressure in the liquid, and the density of the liquid. Calculations of the resonance frequency of an encapsulated air bubble as a function of bubble radius Ro are shown below in Table 1. The parameter Sp is the shell elastic parameter of the bubble, estimated to be 8 N/m. This chart also shows a feature referred to as “scaling.” For example, a 1 μl droplet in an EWD apparatus might have a diameter of about 750 μm. Acoustic streaming within this droplet could be induced by using one or more 15-20 μm diameter trapped air bubbles resonating at 1 MHz. A picoliter droplet would have a diameter of about 10-15 μm. Acoustic microstreaming in the droplet (1) illustrated in FIG. 3 could be induced by a 4 μm diameter bubble oscillating at about 7.4 MHz.

TABLE 1
Resonance frequency vs radius:

| | Sₚ = 8 N/m |
|---|---|
| Rₒ (μm) | fᵣ (MHz) |
| 0.50 | 58.20 |
| 1.00 | 20.70 |
| 1.50 | 11.30 |
| 2.00 | 7.40 |
| 2.50 | 5.30 |
| 3.00 | 4.10 |
| 3.50 | 3.30 |
| 4.00 | 2.70 |
| 4.50 | 2.30 |
| 5.00 | 1.90 |
| 5.50 | 1.70 |
| 6.00 | 1.50 |
| 6.50 | 1.30 |
| 7.00 | 1.20 |
| 7.50 | 1.10 |
| 8.00 | 0.99 |
| 8.50 | 0.91 |
| 9.00 | 0.84 |
| 9.50 | 0.78 |
| 10.00 | 0.72 |

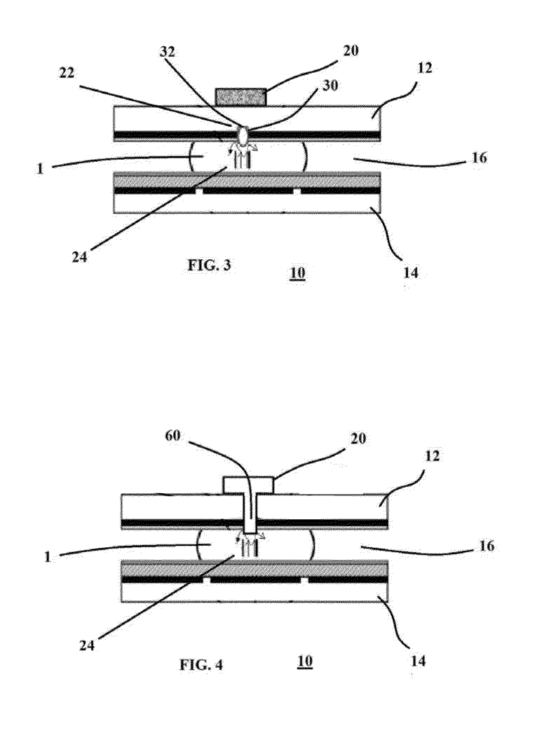

FIG. 4 is a front view of an electrowetting-on-dielectric microfluidic apparatus (10) for cell poration according to one or more embodiments of the present disclosure. The apparatus (10) depicted in FIG. 4 shares many features as the apparatus illustrated in FIG. 2 and FIG. 3, and where the same features are shown, the description of those features as it relates to FIG. 2 and FIG. 3 may also apply to the features of the apparatus (10) shown in FIG. 4. The apparatus (10) illustrated in FIG. 4 includes an immersion transducer (60) extending from the first plate (12) into the flow channel (16) to provide acoustic streaming and sonoporation to cells within a stationary droplet (1) received in the flow channel (16). The immersion transducer (60) is provided for generating vibratory or resonating forces with the droplet (1) in order to impart the fluid flow within the droplet (1) as described herein.

Returning to FIGS. 2 through 4, induced flow within the excited droplet (1) is circular such that the majority of cells in the droplet (1) will be made to pass between the electroporation electrodes (46). Because acoustic streaming has been shown to cause rapid mixing in microfluidic volumes, the flow is robust. Thus, cells that pass through the separation between the electrodes (46) can be exposed to a more controlled, uniform average field, resulting in a reduced applied voltage and more reproducible cell electroporation. In an alternative embodiment, a DC electric field rather than a pulsed field can be used, whereby the flow rate of cells between the electrodes (46) can determine the time of exposure to the field.

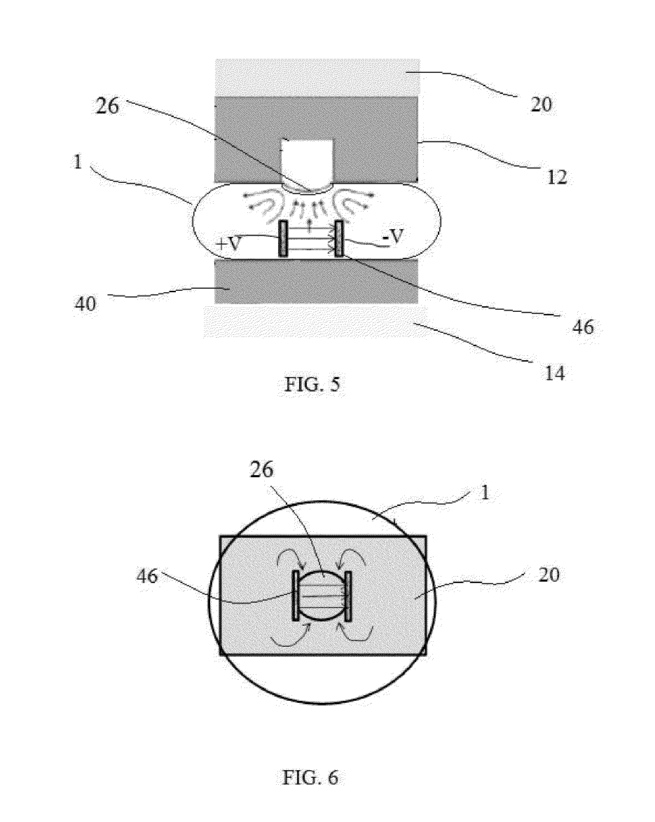

The circular flow of the cells in a droplet (1) is depicted in FIG. 5, which is a front view of the apparatus (10) as illustrated in FIG. 3 and according to one or more embodiments disclosed herein. FIG. 5 is focused on the droplet (1) received in the flow channel (16) of the apparatus (10). FIG. 6 is a top view of the apparatus (10) shown in FIG. 5. Acoustic streaming within the droplet (1) causes liquid flow to occur, thereby directing cells to the surface of the droplet and in close proximity to the resonating deformable membrane (26). The general circular flow motion (which is depicted by arrows in FIGS. 5 and 6) draws cells from the volume of the droplet (1) inward to pass through the electrodes (46) and on to the surface of the droplet (1) close to the resonating deformable membrane (26). Variable levels of acoustic energy and electric field strength between the electrodes (46) can allow for cell poration to be achieved in an optimum way as a balance between sonoporation and electroporation.

In one embodiment of the presently disclosed subject matter, a method is provided for applying an acoustic field to a resilient member in communication with a droplet containing cells to induce an ultrasonic vibration in the resilient member to effect sonoporation of the cells. In the method, the resilient member can be a deformable membrane. In the method, the resilient member can be a bubble. In the method, a droplet can be a stationary droplet. A droplet may be held in a stationary position during sonoporation by the application of a DC or AC voltage on an electrode beneath a droplet.

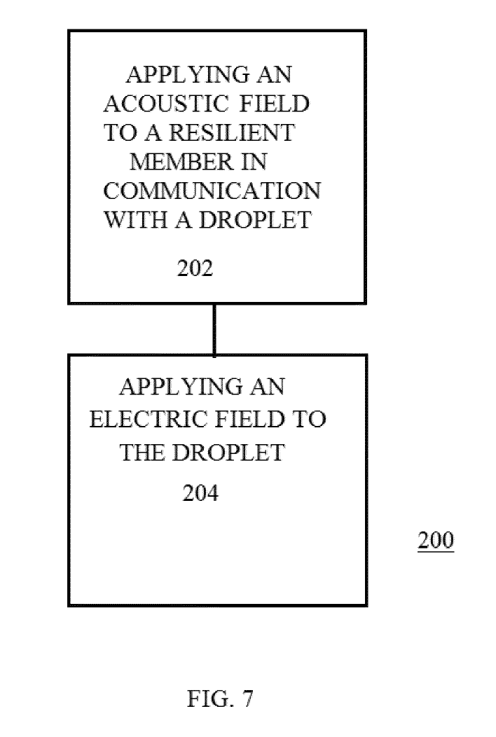

In one embodiment of the presently disclosed subject matter and with further reference to FIG. 7, a method (200) is provided that includes, in a spaced-apart plate arrangement defining a flow channel therebetween, applying an acoustic energy field to a resilient member in communication with a droplet having cells therein to induce an ultrasonic vibration in the resilient member for providing acoustic streaming and sonoporation of the cells (202), and applying an electric field to the droplet having cells therein to effect electroporation of the cells (204). This method (200) may be carried out on any of the embodiments disclosed herein that are configured for these steps.

Providing sonoporation may include providing pulses of energy with an acoustic generator (20).

Additionally disclosed herein is a method for applying sonoporation with an immersion transducer to a droplet as described herein. This method may include applying sonoporation with an immersion transducer that is immersed into a droplet in an electrowetting-apparatus as disclosed and described herein. This sonoporation may be accompanied with an electroporation process as described herein, or may be a stand alone-option.

The methods disclosed herein may be carried out with reference to the system (100). For example, the control module (50) can be configured for controlling the acoustic energy generator (20) to apply energy to the apparatus (10) for providing acoustic streaming and sonoporation to cells within a droplet (1) over a first time period, and for controlling the electric field generator (24) to apply an electric field for providing electroporation to cells within a droplet (1) over a second time period. In one or more embodiments, the first time period and the second time period can be simultaneous. In one or more embodiments, the first time period and the second time period can partially overlap. In one or more embodiments, the first time period and the second time period can be in sequence.

In the method, the resilient member can be a deformable membrane. In the method, the resilient member can be a bubble. In the method, a droplet received in the flow channel can be a stationary droplet. In the method, the electric field can be applied generally perpendicularly to the acoustic energy field and the acoustic streaming can focus the cells within the electric field to provide an electric field thereto.

In the system (100), providing sonoporation can include providing pulses of energy from the acoustic energy generator (20).

In the system (100), providing electroporation can include providing pulses of electric field from the electric field generator (24).

The control module (50) may be configured for controlling the acoustic energy generator (20) and the electric field generator (24) in order to direct each respective generator for the poration process. Additionally, the control module (50) may be in communication with one or more elements configured for advancing a porated droplet away from the area where the poration process occurs and then advancing a new, un-porated droplet into the area where the poration process occurs. The control module (50) may have computer control code installed thereon configured for executing the instructions described herein.

Aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter situation scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the presently described subject matter is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

We claim:

1. An electrowetting-on-dielectric microfluidic apparatus for cell poration comprising:
a first plate;
a second plate spaced-apart from the first plate and defining a flow channel therebetween for receiving a droplet having cells therein;
an acoustic energy generator in communication with the first plate for providing sonoporation to cells within a droplet received in the flow channel; and
a resilient member in communication with the acoustic energy generator and in communication with a droplet received in the flow channel to provide acoustic streaming and sonoporation to cells within a droplet.

2. The apparatus according to claim 1, wherein the resilient member is a deformable membrane carried by the first plate and in communication with a droplet received in the flow channel.

3. The apparatus according to claim 1, wherein the resilient member is a bubble partially enclosed by a recess defined by the first plate, wherein the bubble is in communication with a droplet received in the flow channel.

4. The apparatus according to claim 1, wherein the first plate comprises a hydrophobic layer on a metal film, and wherein the second plate comprises a hydrophobic layer on a dielectric film on a patterned metal electrode.

5. The apparatus according to claim 1, wherein the acoustic energy generator is a piezoelectric disk translator.

6. The apparatus according to claim 1, further including an electric field generator in electrical field communication with the flow channel for providing electroporation to cells within the droplet received in the flow channel.

7. The apparatus according to claim 6, wherein the electric field generator comprises electrodes placed between the first plate and the second plate, and wherein the electrodes are spaced-apart a distance generally corresponding to a width of the resilient member.

8. The apparatus according to claim 6, wherein the acoustic energy generator and the resilient member comprise an immersion transducer extending from the first plate into the flow channel to provide acoustic streaming and sonoporation to cells within a stationary droplet received in the flow channel.

9. The apparatus according to claim 1, wherein a droplet received in the flow channel is a stationary droplet.

10. A system comprising:
an electrowetting-on-dielectric microfluidic apparatus for cell poration that includes:
a first plate;
a second plate spaced-apart from the first plate and defining a flow channel therebetween for receiving a droplet having cells therein;
an acoustic energy generator in communication with the first plate; and
a resilient member in communication with the acoustic energy generator and in communication with a droplet received in the flow channel; and
a control module configured for:
controlling the acoustic energy generator to apply energy to the apparatus for providing acoustic streaming and sonoporation to cells within a droplet.

11. The system according to claim 10, wherein the resilient member is a deformable membrane that is in communication with a droplet received in the flow channel.

12. The system according to claim 10, wherein the resilient member is a bubble partially enclosed by a recess defined by the first plate, wherein the bubble is in communication with a droplet received in the flow channel.

13. The system according to claim 10, wherein the acoustic energy generator and the resilient member comprise an immersion transducer extending from the first plate into the flow channel to provide acoustic streaming and sonoporation to cells within a stationary droplet received in the flow channel.

14. The system according to claim 10, wherein the first plate comprises a hydrophobic layer on a metal film, and wherein the second plate comprises a hydrophobic layer on a dielectric film on a patterned metal electrode.

15. The system according to claim 10, wherein the acoustic energy generator is a piezoelectric disk translator.

16. The system according to claim 10, wherein a droplet received in the flow channel is a stationary droplet.

17. The system according to claim 10, wherein the apparatus further includes an electric field generator in electrical field communication with the flow channel, and wherein the control module is further configured for controlling the electric field generator to apply an electric field for providing electroporation to cells within the droplet.

18. The system according to claim 17, wherein the electric field generator comprises electrodes placed between the first plate and the second plate, and wherein the electrodes are spaced-apart a distance generally corresponding to a width of the resilient member.

19. The system according to claim 18, wherein the control module is configured for:
controlling the acoustic energy generator to apply energy to the apparatus for providing acoustic streaming and sonoporation to cells within a droplet over a first time period; and
controlling the electric field generator to apply an electric field for providing electroporation to cells within a droplet over a second time period.

20. The system according to claim 19, wherein the first time period and the second time period are simultaneous.

21. The system according to claim 19, wherein the first time period and the second time period partially overlap.

22. The system according to claim 19, wherein the first time period and the second time period are in sequence.

23. A method comprising:
in a spaced-apart plate arrangement defining a flow channel therebetween, applying an acoustic energy field to a resilient member in communication with a droplet having cells therein to induce an ultrasonic vibration in the resilient member for providing acoustic streaming and sonoporation of the cells.

24. The method according to claim 23, wherein the resilient member is a deformable membrane.

25. The method according to claim 23, wherein the resilient member is a bubble.

26. The method according to claim 23, further comprising applying an electric field to the droplet having cells therein to effect electroporation of the cells.

27. The method according to claim 26, wherein the electric field is applied generally perpendicularly to the acoustic energy field and the acoustic streaming focuses the cells within the electric field to provide an electric field thereto.

28. The method according to claim 23, wherein a droplet received in the flow channel is a stationary droplet.
